(19) United States
(12) Patent Application Publication
(10) Pub. No.: US 2022/0193002 A1
(43) Pub. Date: Jun. 23, 2022

(54) PROCESS FOR THE PREPARATION OF BAKUCHIOL COMPOSITION SUBSTANTIALLY FREE OF FURANOCOUMARIN IMPURITIES

(71) Applicant: K V Natural Ingredients PVT LTD, Hyderabad (IN)

(72) Inventors: Vijay Kumar Katta, Hyderabad (IN); Ganeswara Rao Mallula, Hyderabad (IN); Rajesh Haldorai, Hyderabad (IN)

(21) Appl. No.: 17/126,810

(22) Filed: Dec. 18, 2020

(51) Int. Cl. A61K 31/05

(52) U.S. Cl. CPC A61K 31/05; A61K 2236/39; A61K 2236/35; A61K 2236/31

(57) ABSTRACT

The present invention relates to a process for preparation of Bakuchiol enriched psoralea extract substantially free of furanocoumarin impurities such as psoralen, isopsoralen and Bakuchicin and composition comprising the same. The process of the present invention uses unpulverized seeds of Psoralea genus of plants such as Psoralea corylifolia and employs novel single column chromatographic separation technique comprising silica gel and aluminium oxide columns clubbed to form single column to obtain a bakuchiol composition which is substantially free of furanocoumarin impurities. The bakuchiol composition is effective for prevention, alleviation, reduction, or treatment of post inflammatory hyper pigmentation (PIH) without any side-effects such as potential phototoxicity, topical irritation, carcinogenicity, and reproductive toxicity associated with furanocoumarin impurities.

### FIELD OF THE INVENTION

The present invention relates to the process for preparation of Bakuchiol enriched psoralea extract composition substantially free of furanocoumarin impurities such as psoralen, isopsoralen and Bakuchicin. The present invention more particularly relates to a composition comprising Bakuchiol which is substantially free of furanocoumarin impurities and that is effective for prevention, alleviation, reduction, or treatment of post inflammatory hyper pigmentation (PIH) without any side-effects such as potential phototoxicity, topical irritation, carcinogenicity, and reproductive toxicity associated with furanocoumarin impurities.

### BACKGROUND OF THE INVENTION

Bakuchiol, 4-[(1E,3S)-3-ethenyl-3,7-dimethylocta-1,6-dienyl]phenol, the structure of which is shown below, is a phenolic compound having a single hydroxyl group on the aromatic ring and an unsaturated hydrocarbon chain. It has been isolated from the seeds of Psoralea corylifolia Linn (Luguminosae) and the aerial part of Psoralea glandulosa L. (Papilionaceae)

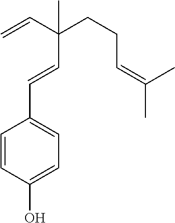 [SMILES: C=CC(C)(/C=C/c1ccc(O)cc1)CCC=C(C)C]

Bakuchiol is a meroterpenee (a chemical compound having a partial terpenoid structure) in the class terpenophenol, from Psoralea corylifolia seeds as botanical source.

Bakuchiol is a useful compound against additives and mouthwashes for preventing and treating dental caries. The cell growth of S. mutans was inhibited in a bakuchiol concentration dependent manner and the growth of S. mutans was completely prevented with 20 μg of bakuchiol/mL. Bakuchiol is an effective vitiligo treatment and also treats related skin diseases and conditions. It shrinks white spots on skin and reduces pigmentation. Bakuchiol is the first natural alternative to Retinol without having any of the negatives associated with retinol. It is photochemically and hydrolytically stable and can be used during the day.

Multiple comparative studies revealed Bakuchiol to be true alternative to retinol. Retinol-like functionality was further confirmed for type I, III and IV collagens and aquaporin 3 by ELISA and histochemistry. Many clinical studies showed significant improvement in lines and wrinkles, pigmentation, elasticity, firmness and overall reduction in photo-damage without usual retinol-associated undesirable effects.

Commercial use of Bakuchiol in a wide-variety of skin care and treatment products has begun and is expanding. Bakuchiol has now become a sought-after ingredient for developing broad aging products.

Bakuchiol works as an anti-inflammatory and an antioxidant so those who suffer from dry, sensitive skin and cannot tolerate a retinol can use in all cosmeticals.

The antibacterial properties of bakuchiol also mean that it is great for those with acne and oily skin.

Bakuchiol has been shown to activate a number of chemical pathways in skin cells that ultimately lead to improved collagen production, decreased collagen breakdown and reduction of melanin (pigment) synthesis. The overall net effect seems to be an improvement in fine lines, wrinkles and pigmentation which are commonly associated with the natural ageing process of the skin.

Bakuchiol is likely the only agent after retinoic acid shown to be effective against multiple pathophysiologic features of acne. Bakuchiol may also have a suppressive effect on melanin-producing cells in skin, which may make it a beneficial ingredient to lighten dark spots or improve uneven skin tone due to sun damage.

Bakuchiol also has antifungal activity, particularly against the type of fungi that cause athlete's foot (medically, Tinea pedis).

EP1881839A2 relates to compositions of bakuchiol and methods of making the same. The EP patent describes large scale extraction of bakuchiol from Psoralea plants using seed powder of Psoralea corylifolia and petroleum ether. It further teaches that the extraction with petroleum ether provides the highest purity bakuchiol in the crude extract with good recovery.

WO2012/105990A1 composition comprising bakuchiol and less than 500 ppm total furanocoumarin impurities. In one embodiment, the composition comprises less than 100 ppm total furanocoumarin impurities. The WO publication teaches extraction of bakuchiol from Psoralea plants using Psoralea corylifolia seed powder and solvent.

US2011/0223267A1 teaches compositions of bakuchiol and methods of making the same. US patent teaches composition of bakuchiol (UP246) having low levels of impurities, particularly furanocoumarin impurities.

EP1714646A1 teaches pharmaceutical composition containing bakuchiol for treating osteoporosis in women.

Extraction, Quantification, and Cytokine Inhibitory Response of Bakuchiol in Psoralea coryfolia Linn; Separations 2020, 7(3), 48; https://doi.org/10.3390/separations7030048. This article teaches extraction of Bakuchiol using powdered seed of P. corylifolia.

Method of Manufacturing Bakuchiol Known in the Literature.

Many manufacturers of Bakuchiol use pulverized seeds for Methanol extraction as the primary extraction followed by hexane partition, hexane soluble paste adsorbed on silica gel and chromatographic separation by using primary column with silica gel and secondary column with aluminium oxide.

In the Primary extraction of Psoralea corylifolia pulverized seeds with Methanol extraction the content of Bakuchiol by HPLC will come around 25-27%, the Content of psoralene and angelicin will be 1.80% & 2.50%.

The time of Extraction and volume of solvents as follows:

I^{st} Extraction: 5.0 Volumes of solvent to the raw material quantity and time of extraction minimum 5.0 Hours.

II^{nd} Extraction: 4.0 Volumes of solvent to the raw material quantity and time of extraction minimum 5.0 Hours.

III^{rd} Extraction; 3.0 Volumes of solvent to the raw material quantity and time of extraction minimum 5.0 Hours.

So total Extraction volume will be 12.0 Volumes to the raw material quantity and total time consumption will be 20-24 hours, with an output Assay 25-27% and % Yield 10-11%.

In the Secondary extraction with Hexane, the primary extracted paste soluble in water and followed by solvent extraction with Hexane with minimum 4-5 volumes to the paste.

And the time of Extraction and volume of solvents as follows:

I^{st} Extraction: 4.0 Volumes to the paste quantity and time of separation minimum 3.0 hours.

II^{nd} Extraction: 3.0 Volumes to the paste quantity and time of separation minimum 3.0 hours.

So total Extraction volume will be 7.0 Volume to the paste quantity and total time consumption will be 6-8 hours, with an output Assay 45-75% and % Yield 4-5%.

The above paste adsorbed on Silica gel with 2.0 volumes to the paste and for purification will follow the two-column process.

I^{st} Column with minimum 10 volumes of Silica Gel chromatography for purification, will get 90.0% assay with an impurity profile using 15 KL of mixture of Hexane and ethyl acetate re-circulated.

II^{nd} Column with minimum 10 volumes of Aluminium oxide column for purification, will get 95.0% assay with an impurity profile up to 200-300 pm using 15 KL of mixture of Hexane and ethyl re-circulated.

Total Column volume will be 30.0 KL of solvent and 22.0 volumes of Silica Gel/Aluminium oxide to the paste quantity and total time consumption will be 7 days, with an output Assay 95-97% and % Yield 2-2.5%.

### Problem Identified by the Present Invention

Bakuchiol is a biologically active natural product having a great deal of potential for use in the prevention and treatment of various diseases and conditions, however there are a number of limitations associated with the use of this compound. Some limitations include its low concentration in natural sources and the presence of co-existing toxic components in the bakuchiol source. The main impurities present in the bakuchiol are Psoralens, also known as furanocoumarins. They are naturally occurring secondary metabolites in Psoralea genus plants (a source of bakuchiol). Furanocoumarins often found co-existing with bakuchiol include Psoralen, Isopsoralen and Bakuchicin.

Because of the toxicity of furanocoumarins, it is important to remove psoralen, isopsoralen and Bakuchicin from bakuchiol compositions intended for treating post inflammatory hyperpigmentation or other conditions.

### Solution Provided by the Present Invention

The present inventors have surprisingly found a process for preparation of composition comprising Bakuchiol substantially free of furanocoumarin impurities such as psoralen, isopsoralen and Bakuchicin.

The present inventors have eliminated the old extraction techniques like solvent extraction and Percolation techniques. The present inventors have found that the ultrasonication technique is the best extraction technique for Bakuchiol.

In the present invention unpulverized seeds are used for extraction, because it has been surprisingly found that the required compound Bakuchiol is present in Pericarp of the seed and due to pulverization (such as in the prior art method) the unwanted junk and furocoumarins impurities are extracted more into the main portion.

In ultrasonication, unpulverized seed for extraction is used to reduce the impurity profile in the final product. In the present invention there is no primary and secondary extraction. The present inventors have identified only one extraction is more sufficient for total yield output.

The present inventors have discovered that Bakuchiol enriched psoralea extract essentially free of furanocoumarin impurities of the present invention can be utilized for prevention, alleviation, reduction or treatment of excess pigmentation. Bakuchiol compositions of the present invention are effective for prevention, alleviation, reduction, or treatment of post inflammatory hyper pigmentation (PIH) without any potential side effects.

Bakuchiol compositions of the present invention are free of furanocoumarin impurities such as psoralen, isopsoralen and Bakuchicin. Thus, the potential phototoxicity, topical irritation, carcinogenicity, and reproductive toxicity associated with said impurities are essentially eliminated.

Additionally, the color of the highly pure furanocoumarin free Bakuchiol compositions of the present invention are light brown or red and it is very stable with respect to both color and composition of the active agent, making them particularly suitable for formulation, storage and cosmetic applications.

### OBJECTS OF THE INVENTION

It is an object of the present invention to provide a process for preparation of Bakuchiol substantially free of furanocoumarin impurities such as psoralen, isopsoralen and Bakuchicin.

It is another object of the present invention to provide Bakuchiol enriched psoralea extract essentially free of furanocoumarin impurities which can be utilized for prevention, alleviation, reduction or treatment of excess pigmentation without any potential side effects.

It is another object of the present invention to provide Bakuchiol compositions free of furanocoumarin impurities such as psoralen, isopsoralen and Bakuchicin which eliminate the potential phototoxicity, topical irritation, carcinogenicity, and reproductive toxicity associated with said impurities.

### SUMMARY OF THE INVENTION

According to an aspect of the present invention there is provided a method for the preparation of a composition comprising Bakuchiol that is substantially free of furanocoumarin impurities, said method comprising the steps of:
a. Extracting unpulverized seeds of Psoralea genus of plants with petroleum ether through ultrasonication technique;
b. Purifying extract obtained in step (a) by single column chromatographic separation technique comprising silica gel and aluminium oxide columns clubbed into a single column to obtain a bakuchiol composition which is free of furanocoumarin impurities.

According to another aspect of the present invention there is provided a Bakuchiol enriched psoralea extract substantially free of furanocoumarin impurities such as Psoralen, Isopsoralen and Bakuchicin.

### DETAILED DESCRIPTION OF THE INVENTION

The following description is provided to assist in a comprehensive understanding of exemplary embodiments of the invention. It includes various specific details to assist in that understanding but these are to be regarded as merely exemplary.

Features that are described and/or illustrated with respect to one embodiment may be used in the same way or in a similar way in one or more other embodiments and/or in combination with or instead of the features of the other embodiments.

The present invention relates to process for preparation of Bakuchiol enriched psoralea extract essentially free of furanocoumarin impurities such as psoralen, isopsoralen and Bakuchicin. Thus, the potential phototoxicity, topical irritation, carcinogenicity, and reproductive toxicity associated with these compounds are essentially eliminated. The present invention more particularly relates to Bakuchiol compositions that is effective for prevention, alleviation, reduction, or treatment of post inflammatory hyper pigmentation (PIH).

“Prevention” in the context of the present invention refers to prophylactic methods which hinder or stop the occurrence of a particular condition, for example PIH.

“Alleviation” in the context of the present invention refers to lessening or mitigating the effects or symptoms of a particular condition, for example PIH.

“Reduction” in the context of the present invention refers to decreasing the effects or symptoms of a particular condition, for example PIH.

“Treatment” in the context of the present invention refers to techniques or methods intended to improve the symptoms of decrease or stop the occurrence of a particular condition, for example PIH.

“Impurity” includes any substance that is not desired in the bakuchiol composition, typically resulting from the isolation of bakuchiol from natural sources. The term impurity includes, but is not limited to furanocoumarin compounds including, but not limited to, psoralen, isopsoralen and other coumarin type impurities.

In an embodiment there is provided a method for the preparation of a composition comprising Bakuchiol that is substantially free of furanocoumarin impurities said method comprising the steps of:
a. Extracting unpulverized seeds of Psoralea genus of plants with petroleum ether through ultrasonication technique;
b. Purifying extract obtained in step (a) by single column chromatographic separation technique comprising silica gel and aluminium oxide columns clubbed into a single column to obtain a bakuchiol composition which is free of furanocoumarin impurities.

In another embodiment, the bakuchiol is extracted from Psoralea genus of plants, for example, Psoralea corylifolia L. (Luguminosae) or Psoralea glandulosa L. (Papilionaceae).

In a preferred embodiment Bakuchiol is extracted from seed of Psoralea corylifolia.

The purity of Bakuchiol obtained by the process of the present invention is not less than 99% and with impurity profile not more than 20.0 ppm.

In yet another embodiment there is provided a Bakuchiol enriched psoralea extract substantially free of furanocoumarin impurities such as Psoralen, Isopsoralen and Bakuchicin. The purity of Bakuchiol in the said extract is not less than 99% and content of impurity not more than 20.0 ppm.

### Examples

The present invention will now be explained in detail by reference to the following formulation examples and a test example, which should not be construed as limiting the scope of the present invention.

Manufacturing Process:

Extraction:

In the Ultrasonication extraction of Psoralea corylifolia unpulverized seeds with PETROLEUM ETHER 60-80 extraction the content of Bakuchiol by HPLC will come around 65-67%, the Content of psoralene and angelicin will be 0.80% & 0.50%. In regular solvent extractions the limits will be 1.5-2.0%.

Initially we have processed with the coarse powder of P. corylifolia L. It was extracted with different solvents (ethanol, methanol, acetone, petroleum ether, and dichloromethane) in Raw material:solvent ratio of 1:10 volumes by ultrasonication method for 45 min with input power of 220 V and heating power of 550 W, equipped with time and temperature controller. The extracts obtained were concentrated under reduced pressure to obtain brownish colored viscous liquid. The extract was evaporated to dry to get brownish extract, which contains Bakuchiol by HPLC assay 28-30.0% Assay. That results almost same as regular extraction process.

After that we have invented a process which contains the unpulverized seeds extracted with a high-intensity probe system (Model: UIP2000dht) of 2000 W and 20 kHz from Heilscher Ultrasound Technology. Ultrasonic probe was immersed in a seed-solvent mixture. Raw material:solvent ratio of 1:4 volumes. Temperature was maintained between 40-60° C. Sample was treated with ultrasound power range 60-90% with amplitude 70-90% for 15 min. The extracts obtained were concentrated under reduced pressure to obtain light brownish colored viscous liquid. The extract was evaporated to dry to get a light brownish extract, which contains Bakuchiol by HPLC assay 65-68.0% Assay. That results almost double to the regular extraction process

The Time of Extraction and Volume of Solvents are as Follows:

Extraction: 4.0 Volumes of solvent to the raw material quantity and time of extraction maximum 15.0 minutes.

Total Extraction solvent volume will be 4.0 Volumes to the raw material quantity and total time consumption will be 15.0 minutes, with an output Assay 65-67% and % Yield 5.5-6.0 in a single extraction.

The extraction process of the present invention eliminates the 12.0 volumes of alcohol consumption and 7.0 volumes of Hexane consumption in the process and reduces the 30.0 hours of time cycle.

Purification:

The Novel Column chromatographic separation technique in the purification step is used in the present invention. The present inventors have clubbed the Silica Gel and Aluminium oxide columns in a single column in calculated proportions based on NLT 65% assay extracted material.

65-67% assay paste on 1.5 volumes of silica Gel 100-200 #Mesh and chromatographed over 3.5 volumes of calculated proportionate of Silica Gel and Aluminum oxide as a separation column, the solvent used for the column was only 10.0 KL of Hexane and Ethyl acetate solvent re-circulated.

One single column with total 5.0 volumes of separation Silica Gel and Aluminum oxide reduces the 10.0 volumes of Silica Gel and 10.0 volumes of Aluminum oxide, which will be a great benefit for the environment, because the Silica Gel and Aluminum oxide are not reused products, both the products are effluents only. The process of the present invention reduces the effluent load.

In the present invention by using invented column process by taking Ultrasonic extracted material, an Assay of Bakuchiol NLT 99.0% with and impurity profile NMT 20.0 ppm is obtained [Content of Psoralene and Angelicin will be 2.0 ppm & 0.1 ppm]

The present invention reduces the huge alcohol use in extraction process as well as time of the process which includes pulverization, extraction, filtration and alcohol distillation which in turn reduces the energy consumption, manpower and working hours with better assay [67% Instead of 27%], and the product is free from Alcohol [Methanol/Ethanol] traces.

And in continuation the separation step also reduces the Silica Gel and Aluminum oxide quantities as well as solvent consumption, there is huge solvent consumption for older two column process which includes solvent distillation and loss etc. In single column process of the present invention the solvent consumption of solvent is reduced and higher assay with lesser impurity load is obtained.

### EXAMPLES

The present invention will now be explained in detail by reference to the following examples which should not be construed as limiting the scope of the present invention.

### Example 1

1000.0 Kg Psoralea Corylifolia Linn unpulverized seeds were extracted with 4000.0 Ltrs Petroleum ether using a high-intensity probe system (Model: UIP2000dht) of 2000 W and 20 kHz from Heilscher Ultrasound Technology. Ultrasonic probe was immersed in a seed-solvent mixture, Temperature was maintained between 40-60° C. Sample was treated with ultrasound power range 60-90% with amplitude 70-90% for 15 min.
The above extract was evaporated to dry to get a light brownish extract, weight 50.10 Kg, which contains Bakuchiol by HPLC assay 67.29% Assay and Content of Psoralene and Angelicin are 0.49% and 0.62% respectively.
The 50.0 Kg crude light brown colored paste was adsorbed on 75.0 Kg of Silica gel 100-200 Mesh #, and chromatographed over the column contains a total 175.0 Kg of Silica gel and aluminium oxide.
As per TLC, collected the Bakuchiol pure fractions and distilled of the fractions to get pure honey colored product Qty: 19.5 Kg with an Assay 99.20% and content of Psoralene and Angelicin are 0.02 ppm % & 0.01 ppm % respectively.

The product of the present invention [NeuvaChiol™ [Bakuchiol 99%]] contains Bakuchiol with a purity 99.90% and furanocoumarin impurities limit was NMT 20.0 ppm and this product is free from Additives/Excipients and the Assay limit was 99.0%. The Bakuchiol known product offer only 99.0% purity and their assay is NMT 90.0%. The product of the present invention have the Assay limit NLT 99.0%, % Moisture by KF [USP<921>1.0%] to make 100.0% composition.

We claim:

1. A method for the preparation of a composition comprising Bakuchiol that is substantially free of furanocoumarin impurities said method comprising the steps of:
a. Extracting unpulverized seeds of Psoralea genus of plants with petroleum ether through ultrasonication technique;
b. Purifying extract obtained in step (a) by single column chromatographic separation technique comprising of silica gel and aluminium oxide columns clubbed into a single column to obtain a bakuchiol composition which is free of furanocoumarin impurities.

2. The method for the preparation of a composition comprising Bakuchiol as claimed in claim 1 wherein the plant is Psoralea corylifolia Linn.

3. The method for the preparation of a composition comprising Bakuchiol as claimed in claim 1 wherein the time for extraction in step (a) is in the range from 15 to 45 min.

4. The method for the preparation of a composition comprising Bakuchiol as claimed in claim 1 wherein the purity of Bakuchiol is not less than 99% with impurity profile not more than 20.0 ppm.

5. A Bakuchiol composition substantially free of furanocoumarin impurities such as Psoralen, Isopsoralen and Bakuchicin.

6. The Bakuchiol composition as claimed in claim 5 wherein the purity of Bakuchiol is not less than 99.0% and Assay of the main ingredient Bakuchiol by HPLC is also not less than 99.0%.

7. The Bakuchiol composition as claimed in claim 5 contains Bakuchiol with a purity 99.90% and Assay of the main ingredient Bakuchiol by HPLC is also not less than 99.0% and furanocoumarin impurities limit was not more than 20.0 ppm and the said Bakuchiol composition is free from Additives and Excipients.

8. The Bakuchiol composition as claimed in claim 7 wherein the Assay limit is not less than 99.0%.
